(19) United States
(12) Patent Application Publication
(10) Pub. No.: US 2012/0009263 A1
(43) Pub. Date: Jan. 12, 2012

(54) GRANULAR DELIVERY SYSTEM

(76) Inventors: Christopher Gregson, Princeton, NJ (US); Matthew Sillick, Hamilton, NJ (US)

(21) Appl. No.: 13/258,913

(22) PCT Filed: May 12, 2010
(86) PCT No.: PCT/IB10/52099

§ 371 Date: Sep. 22, 2011

Related U.S. Application Data
(60) Provisional application No. 61/177,927, filed on May 13, 2009.

(30) Foreign Application Priority Data
May 20, 2009 (EP) 09160740.8

(51) Int. Cl. A61K 9/00; A23G 4/10; B01J 13/22; A23L 2/52; A23L 1/00; B29B 9/16; A23P 1/04; A23L 1/30

(52) U.S. Cl. 424/488; 426/96; 426/3; 426/648; 426/590; 426/658; 264/141; 427/2.14

(57) ABSTRACT

A granular delivery system and a method of preparing the same by creating a melt emulsion having a continuous phase and a dispersed active, wherein the continuous phase includes trehalose and a hydrogenated starch hydrolysate having number average degree of polymerization, DPn, of between 5 and 100, or a number average molecular weight, Mn of between 800 and 16000 Da, forcing the melt emulsion through an die or orifice to form an extrudate, cooling and granulating the extrudate to form granules of the delivery system and optionally drying the granules.

### TECHNICAL FIELD

The present invention relates to a granular delivery system. It also relates to a process for preparing such a granular delivery system.

### BACKGROUND AND PRIOR ART

Delivery systems or encapsulation systems are used in various industries to protect active ingredients. For instance, in the food industry they are often used to protect flavors, in particular against losses of volatile components (i) during storage prior to incorporation into the food products, (ii) during mixing of the flavor component with the other food ingredients, (iii) during food processing, such as cooking and baking, (iv) during transportation and storage and (v) during the preparation of the food product by the end-consumer.

Similarly, in the nutraceutical industry, they are often used to protect oxygen-sensitive active material, such as fish oils rich in polyunsaturated fatty acids, by providing an oxygen barrier around the material.

In the fragrance industry it is known to encapsulate perfumes for use in homecare products such as fabric conditioners. This enables the perfumes to be deposited onto the fabric without being degraded and released gradually over a longer period than when unencapsulated.

Due to the importance of delivery systems across a broad array of fields, it is not surprising that various different types of delivery system exist. Among the different systems known in the art, extrusion methods typically rely on the use of carbohydrate matrix materials which are heated to a molten state and combined with the active ingredient(s), such as an oxygen sensitive oil, before extruding and quenching the extruded mass to form a glass which protects the active ingredient(s). Such extrusion methods are typically referred to as “melt-extrusion”.

One significant example of the prior art disclosure in this field is in the patent U.S. Pat. No. 3,704,137 which describes an essential oil composition formed by mixing oil with an antioxidant, separately mixing water, sucrose and hydrolyzed cereal solids with DE below 20, emulsifying the two mixtures together, extruding the resulting mixture in the form of rods into a solvent, removing the excess solvent and finally, adding an anti-caking agent.

Further examples are described in U.S. Pat. No. 4,610,890 and U.S. Pat. No. 4,707,367 in which compositions are prepared by forming an aqueous solution containing a sugar, a starch hydrolysate and an emulsifier. An essential oil is blended with the aqueous solution in a closed vessel under controlled pressure to form a homogeneous melt, which is then extruded into a relatively cold solvent, dried and combined with an anti-caking agent.

Extruded granular delivery systems formed by melt-extrusion typically comprise a matrix material or carrier material for a material, product or ingredient that is encapsulated. The matrix material is often described as “viscous” or “rubbery” during the extrusion process and “glassy” in the finished product. The temperature at which the matrix material transitions between the glassy and rubbery states is known as the glass transition temperature (referred to herein as “Tg”). A protocol for measuring the Tg of a material such as a matrix material is given in the publication Maltodextrin molecular weight distribution influence on the glass transition temperature and viscosity in aqueous solutions F. Avaltroni, P. E. Bouquerand and V. Normand Carbohydrate Polymers, 2004, Volume 58, Issue 3, 323-334.

It is recognised by many experts in the field that, in the glassy state, i.e. at temperatures below the Tg, all molecular translation is halted and it is this which provides effective entrapping of the flavor volatiles and prevention of other chemical events such as oxidation. Conversely, at temperatures above the Tg, the encapsulation of materials, products and ingredients is ineffective since the rubbery matrix allows the material to be encapsulated to leak.

Thus, the higher the Tg, the more stable the final product is upon storage. However, a higher Tg is known to render more difficult the extrusion conditions since the temperature in the extruder must be raised even higher to allow the mixture to flow under extrusion conditions and to enable the matrix and material to be encapsulated to mix intimately. Such high temperatures can have a variety of adverse effects: loss of volatile materials; unwanted reactions between matrix (encapsulating) ingredients and the active material; and increased energy requirements and consequential manufacturing cost.

Balancing the needs for both a sufficiently low viscosity during extrusion and a sufficiently solid glass after extrusion is a problem especially associated with melt-extrusion processes and products since matrices that do not require such a melt processing step are not exposed to these difficulties.

Accordingly, it would be desirable to provide a granular delivery system having a high Tg but which remains readily or easily processed under extrusion conditions.

In U.S. Pat. No. 6,607,771, trehalose is mentioned as part of a list of sugars that can be present in extruded capsules. However, there is neither explicit preference given for nor any examples of this material. Instead, preference is given to maltodextrin which, as explained above, does not disclose the surprising benefits that are associated with trehalose.

Trehalose is also mentioned in U.S. Pat. No. 6,187,351 as part of a list of sugars that can be used in extruded capsules. Again, there is no explicit preference given for this material, and there are no examples using trehalose. Instead, mixtures of maltodextrin and corn syrup solids are disclosed in the examples and, as explained above, this does not disclose the surprising benefits that are associated with trehalose.

Trehalose is referred to in yet another document, U.S. Pat. No. 5,603,971, as part of a list of sugars that can be used in extruded capsules. Once again, there is no explicit preference given for this material, and there are no examples describing its use. Instead, the examples disclose mixtures of maltodextrin and corn syrup solids.

WO-A1-2004/017762 (Unilever) discloses, in example 1, bouillon cubes prepared by mixing together 210 g matrix material, 70 g water, 60 g salt and 31 g monosodium glutamate. In example 1B, the matrix material consists of 210 g trehalose. The mixture is heated, flavor added and the resulting mixture poured into molds having a size of 2 cm length, 2 cm depth and 2 cm width (as used for preparing ice cubes), upon which the molds are cooled. The resulting cube crystallises significantly on cooling and therefore does not comprise a fully glassy carbohydrate matrix suitable for flavor encapsulation.

In “Physicochemical characterization and oxidative stability of fish oil encapsulated in an amorphous matrix containing trehalose”, Drusch et al, Food Research International 39 (2006) 807-815, there is described the use of trehalose in the context of spray drying. Although this work is focused on the use of trehalose for microencapsulation, it does not mention melt-extrusion nor the benefit of trehalose on the viscosity of melts in relation to its Tg.

EP-A1-1504675 discloses the use of trehalose with any number of other carbohydrate compounds in an extrusion process in the formation of a powder. Any conventional carbohydrate, such as maltodextrin, is referred to as suitable for use in the product.

It is known that the high temperatures associated with extrusion can cause sugars to brown undesirably through reactions such as caramelisation. To the best of our knowledge, none of the prior art documents addresses this issue.

Accordingly, the present invention seeks to resolve one or more of these issues.

### SUMMARY OF THE INVENTION

Surprisingly, it has now been found that a granular delivery system comprising trehalose and HSH, as defined below, addresses one or more of the problems identified above.

Thus, according to the present invention there is provided a granular, extruded delivery system comprising a glassy carbohydrate matrix and a material, product or ingredient encapsulated therein, the matrix comprising:

(i) a hydrogenated starch hydrolysate having number average degree of polymerization, DPn, of between 5 and 100, or a number average molecular weight, Mn of between 800 and 16000 Da, and

(ii) trehalose.

The invention further relates to a method of preparing a granular delivery system comprising the steps of:

(i) creating a melt emulsion comprised of a continuous phase and a dispersed phase comprising an active ingredient, the continuous phase comprising trehalose and a hydrogenated starch hydrolysate having number average degree of polymerization, DPn, of between 5 and 100, or a number average molecular weight, Mn of between 800 and 16000 Da,

(ii) forcing the melt emulsion through an die or orifice to form an extrudate,

(iii) cooling and granulating the extrudate,

(iv) optionally drying the granules.

### DETAILED DESCRIPTION

The granular delivery system of the invention comprises a matrix formed of trehalose together with one or more hydrogenated starch hydrolysates having number average degree of polymerization, DPn, of between 5 and 100, or a number average molecular weight, Mn of between 800 and 16000 Da (referred to herein as “HSH”).

HSH includes hydrogenated glucose syrups, maltitol syrups, and sorbitol syrups, and is a family of products found in a wide variety of foods. HSH is produced by the partial hydrolysis of corn, wheat, or potato starch with the subsequent hydrogenation of the hydrolysate at high temperature under pressure. By varying the conditions and extent of the hydrolysis, the relative occurrence of various mono-, di- , oligo- and polymeric hydrogenated saccharides in the resulting product can be obtained.

Hydrogenated mono-, di-, oligo- and polysaccharides are characterized by the degree of polymerization (DP) or molecular weight (M). For example, hydrogenated monosaccharides have a DP of 1 and an M of 182 Da and hydrogenated disaccharides have a DP of 2 and an M of 344 Da. As HSH has a distribution of molecular weight fractions, a suitable average is often calculated. A convenient averaging scheme is the number average. The number average degree of polymerization, DPn, and the number average molecular weight, Mn, may be determined by routine HPLC analysis or cryoscopy (depression of freezing point), also called freezing point osmometry.

For the purposes of the present invention, the term HSH can be applied to any polyol produced by the hydrogenation of the saccharide products of starch hydrolysis. In practice, however, certain polyols such as sorbitol, mannitol, and maltitol are referred to by their common chemical names. The term HSH is more commonly used to describe the broad group of polyols that contain substantial quantities of hydrogenated oligo- and poly-saccharides in addition to any monomeric (such as sorbitol and mannitol) or dimeric (such as maltitol) polyols. For the purposes of the present invention, HSH is defined as a hydrogenated starch hydrolysate having number average DPn between 5 and 100. More preferably, the DPn is between 6 and 60. Even more preferably the DPn is between 6 and 40, most preferably between 6 and 20. The HSH may have a number average molecular weight, Mn of between 800 and 16000 Da, more preferably between 1000 and 3500 Da.

HSH excludes conventional maltodextrins having a DE of from 5 to 20. DE as used in this context refers to the percentage of reducing sugars (dry basis) in a product calculated as dextrose. Maltodextrins are usually produced by the action of the enzyme a-amylase and/or strong acids on gelatinized starch. Maltodextrin contains a range of nutritive non-sweet polysaccharides with a distribution of molecular weights where the anhydroglucose units are linked predominantly by 1,4 bonds.

The use of HSH in the products of the present invention is particularly advantageous since it is found that, in combination with trehalose, HSH provides a very low reactivity matrix which is substantially less sensitive to pH changes and so can be used in a wider variety of foodstuffs and beverages or for the encapsulation of a wider range of ingredients than for compositions comprising conventional maltodextrin. Such a mixture is also found to be less susceptible to undesirable browning reactions, such as Maillard reactions, during the extrusion process.

In the matrix the amount of HSH is preferably from 90 to 35 by weight, based on the total dry weight of the matrix, more preferably from 70 to 40, most preferably from 60 to 45.

The matrix further comprises trehalose. Trehalose, also known as mycose, is a natural alpha-linked disaccharide formed by an α,α-1,1-glucoside bond between two α-glucose units and is commercially available, typically as the crystalline dihydrate, from a wide variety of suppliers. For instance, a suitable commercially available trehalose product is Ascend and Treha (tradenames) available from Cargill.

In the matrix, the amount of trehalose is preferably from 10 to 65% by weight, based on the total dry weight of the matrix, more preferably from 30 to 60%, most preferably from 40 to 55%. Outside of these ranges, certain disadvantages become apparent. For instance, at lower levels the benefit of reduced viscosity enabling easier extrusion is reduced significantly, whilst at higher levels the risk of crystallization of the matrix increases leading to a reduction or loss of the glassy structure around the encapsulated material. The glassy structure is highly desirable as it enables excellent retention of volatile encapsulated materials.

Trehalose is also found to provide a more stabilised structure in low pH systems than that obtained using conventional sugars, especially sucrose. This allows the matrix to be used in a wider variety of end-products than conventional matrices.

Most importantly, the use of trehalose has the benefit of providing a high Tg for the matrix whilst, very surprisingly, enabling extrusion at lower than expected temperatures, when compared to systems comprising, for example, sucrose. That is, trehalose provides an unexpected increase in the stability of the granule without detrimentally affecting the processing conditions.

The HSH and trehalose can be mixed according to any suitable method. For instance, they can simply be premixed in a hopper without any special equipment.

Advantageously, since the crystalline dihydrate of trehalose has a melting point of about 98° C., it can be melted directly in a typical extruder. By contrast, conventionally used sugars, and especially sucrose which has a melting point of about 185° C., cannot be directly melted in this manner and instead require an additional processing step, such as dissolution in water.

An acid, such as ascorbic acid may advantageously be present since it is found that, in the presence of such a material, the Tg of the hydrogenated starch hydrolysate-trehalose matrix remains very high, e.g. it preferably remains above room temperature whereas that of the corresponding hydrogenated starch hydrolysate-sucrose matrix is depressed significantly below room temperature (rendering it unstable upon storage), even in the presence of the acid.

The active ingredient to be encapsulated can designate a single hydrophobic compound or a composition, such as flavors, fragrances, pharmaceuticals, nutraceuticals or other ingredients, which one wishes to encapsulate.

Preferably, the active ingredient is a volatile or labile flavoring, perfuming or nutraceutical ingredients or composition. Preferably, the active ingredient is a hydrophobic liquid, which is soluble in organic solvents but only very weakly soluble in water. More particularly, a flavoring, perfuming or nutraceutical ingredient or composition encapsulated according to the invention is preferably characterised by a Hildebrand solubility parameter smaller than 30 [MPa]^{1/2}. The aqueous incompatibility of most oily liquids can be in fact expressed by means of Hildebrand's solubility parameter δ which is generally below 25 [MPa]^{1/2}, while for water the same parameter is of 48 [MPa]^{1/2}, and of 15-16 [MPa]^{1/2} for alkanes. This parameter provides a useful polarity scale correlated to the cohesive energy density of molecules. For spontaneous mixing to occur, the difference in δ of the molecules to be mixed must be kept to a minimum. The Handbook of Solubility Parameters (ed. A. F. M. Barton, CRC Press, Bocca Raton, 1991) gives a list of δ values for many chemicals as well as recommended group contribution methods allowing to calculate δ values for complex chemical structures.

The phrase “flavor or fragrance compound or composition” as used herein, thus defines a variety of flavor and fragrance materials of both natural and synthetic origin. They include single compounds and mixtures. Natural extracts can also be encapsulated in the extrudate; these include e.g. citrus extracts, such as lemon, orange, lime, grapefruit or mandarin oils, or essential oils of spices, amongst other. Particularly preferred active materials in this class for encapsulation are flavor compositions containing labile and reactive ingredients such as berry and dairy flavors.

Further specific examples of such flavor and perfume components may be found in the current literature, e.g. in Perfume and Flavour Chemicals, 1969, by S. Arctander, Montclair N.J. (USA) ; Fenaroli's Handbook of Flavour Ingredients, CRC Press or Synthetic Food Adjuncts by M. B. Jacobs, van Nostrand Co., Inc. They are well-known to the person skilled in the art of perfuming, flavoring and/or aromatizing consumer products, i.e. of imparting an odour or taste to a consumer product.

An important class of oxygen-sensitive active materials that can be encapsulated in the delivery system of the present invention are “oils rich in polyunsaturated fatty acids”, also referred to herein as “oils rich in PUFA's”. These include, but are not limited to, oils of any different origins such as fish or algae. It is also possible that these oils are enriched via different methods such as molecular distillation, a process through which the concentration of selected fatty acids may be increased. Particularly preferred compositions for encapsulation are nutraceutical compositions containing polyunsaturated fatty acids and esters thereof.

Specific oils rich in PUFA's for use in the present delivery system include eicosapentanoic acid (EPA), docosahexanoic acid (DHA), arachidonic acid (ARA), and a mixture of at least two thereof.

The encapsulated material is preferably present in the granular delivery system in an amount ranging from about 5% to about 40% by weight, based on the total weight of the delivery system.

A viscosity modifier may be present in the granular delivery system in order to aid the extrusion process. The viscosity modifier may be added at any time prior to or during the extrusion process. Examples of suitable viscosity modifiers include ethyl cellulose (e.g. the Ethocel range from Dow Chemicals), hydrophobic silicas, silicone oils, high viscosity triglycerides, organophilic clay, oil soluble polymers, high viscosity mineral oil (paraffinic and naphthenic liquid hydrocarbons), oleum treated and hydrogenated mineral oils, petroleum jelly, microcrystalline waxes and paraffin waxes.

The preferred viscosity modifier is ethyl cellulose since it is found to provide the additional advantage of having surface active properties that lower the interfacial tension between a material to be encapsulated and the matrix materials, thereby lowering the energy required during the extrusion process.

Preferably, the molecular weight of the ethyl cellulose is preferably within the range of from 50,000 to 2,000,000, more preferably from 75,000 to 1,500,000, most preferably from 100,000 to 1,250,000.

Preferably, the viscosity of the modified cellulose ether is from 50 mPa·s to 1,000 mPa·s, more preferably 75 mPa·s to 750 mPa·s, most preferably 100 mPa·s to 500 mPa·s, measured as a 5% solution based on 80% toluene 20% ethanol, at 25° C. in an Ubbelohde viscometer.

The amount of viscosity modifier required depends on the nature of the viscosity modifier and the material to be encapsulated and can be adjusted accordingly by the skilled person to achieve the correct viscosity.

It may be desirable to include one or more additional ingredients to increase the solubility or dispersibility of the viscosity modifier.

Optionally and advantageously, an emulsifier may be added to the mixture. This is found to decrease the interfacial tension between the oil and melt phases thereby lowering the energy for droplet formation. Additionally, it can stabilize the droplets once formed. Examples of suitable emulsifiers include lecithin, modified lecithins such as lyso-phospholipids, DATEM, mono-diglycerides of fatty acids, sucrose esters of fatty acids, OSA starch, sodium octenyl succinate modified starch, gum Arabic, citric acid esters of fatty acids, and other suitable emulsifiers as cited in reference texts such as Food Emulsifiers And Their Applications, 1997, edited by G. L. Hasenhuettl and R. W. Hartel.

Lecithins and modified lecithins are particularly preferred emulsifiers for use in the present invention. Suitable examples include, but are not limited to soy lecithin (such as Yelkin SS, ex Archer Daniel Midlands) and lyso-phospholipids (such as Verolec HE60, ex Lasenor).

Other optional ingredients can be present in the matrix. For instance, water may be present to modify the characteristics of the HSH.

Similarly, adjuvants such as food grade colorants can also be added in a generally known manner, to the extrudable mixtures of the invention so as to provide colored delivery systems.

If desired, an anticaking agent can be added to the extruded product to reduce the risk of the granules from sticking to one another.

The granular delivery system preferably comprises particles of substantially uniform granulometry. Preferably the average particle size, based on the mean diameter, of the granules is from 200 to 4000 microns. An extruded delivery system can be formed into granules by a variety of processes, all of which are known to the person skilled in the art.

The granular delivery system can be used to enhance a variety of products. For instance, it can deliver an active ingredient to edible compositions, pharmaceutical compositions, nutraceutical compositions, chewing-gum or toothpaste.

The matrix is found to be stable at both acidic and alkaline pH's and is thus more versatile than traditional melt-extruded glassy carbohydrate systems that are not as stable at acidic pH's. It is also different from other trehalose/non-HSH carbohydrate matrices that are more stable at acidic pH's but not as stable at alkaline pH's. Thus, the matrix may desirably be used in a foodstuff having a pH of 6 or less, more preferably 5 or less. Nevertheless, the matrix is also suitable for use in a foodstuff having an a pH of, for example up to 8 or even up to 10, such as anti-acid tablets or effervescent formulations.

Its use in other products is envisaged but are not named here for the sake of brevity. If the active ingredient is a flavors oil, it can be advantageously used to impart or modify the organoleptic properties of a great variety of edible products, i.e. foods, beverages, pharmaceuticals and the like. In a general manner, they enhance the typical organoleptic effect of the corresponding unextruded flavor material.

Where the active material is an oil rich in polyunsaturated fatty acids or a nutraceutical composition comprising such an oil, it can be provided in any foodstuff where health benefits are desired. In such products, a further advantage of the present delivery system is that it can mask the flavor of the oil rich in polyunsaturated fatty acids, which may not be compatible with the flavor of the foodstuff into which it is incorporated.

The concentrations in which the delivery system can be incorporated in such consumer products vary in a wide range of values, which are dependent on the nature of the consumer product and that of the particular delivery system of the invention used.

Typical concentrations, to be taken strictly by way of example, are comprised in a range of values as wide as from a few p.p.m. (parts per million) up to 5 or even 10% of the weight of the flavoring composition or finished consumer product into which they are included.

The granular delivery system of the invention is prepared by extrusion. It can be formed using any current extruder typically used according to prior known “wet extrusion” or “dry blend” (also called “flash-flow”) techniques, the latter requiring feeding of a melt of an originally mainly solid mass into the extruder, and the former requiring the extrusion of a mainly fluid mass melt resulting from the prior solution of the matrix in a suitable solvent.

By extrusion methods we mean here methods according to which, typically, the components which form the glassy carbohydrate matrix, the material that is to be encapsulated and, optionally a plasticizer and an emulsifier, in the form of a melt-emulsion, are forced through a die and then quenched to form a solid product having the encapsulated material dispersed therein. The terms “melt” and “melt-emulsion” are used interchangeably in the text and both denote a liquid matrix as a continuous phase with particles, preferably hydrophobic particles, dispersed therein as the dispersed phase.

As used herein, the term “particles” means both solid articles and liquid droplets.

The melt can be formed in any way known in the art. This includes the heating of matrix ingredients to a temperature which allows the formation of an homogeneous melt, for example in a single or twin screw extruder. An alternative example is the dissolution of matrix ingredients in a solvent, preferably water, followed by the removal of some or all of this solvent by evaporation.

The extruded product can then be formed into granules by any suitable means. For instance, it can be chopped whilst it is still in a plastic state (melt granulation or wet granulation techniques), or it can be cooled in a liquid solvent to form the extruded solid, the shape and size of which can be adjusted as a function of the extrusion parameters before being ground, pulverised or the like.

If desired the die orifice itself can be equipped with a cutter-knife or any other cutting device. Alternatively, the cutting device can be provided separately downstream from the die orifice.

### EXAMPLES

The invention will now be described in further detail by way of the following examples.

### Example 1

### Preparation of a Granular Delivery System

A granular delivery system was prepared using the following ingredients in the amounts shown:

TABLE 1

| Ingredient | Grams |
|---|---|
| Hydrogenated Starch Hydrolysate (1) | 1300 |
| Trehalose dihydrate (2) | 1422 (1300 on an anhydrous basis) |
| Ascorbic Acid | 30 |
| Cold Pressed Orange Oil (3) | 300 |
| Soya lecithin (4) | 30 |
| Water | 700 |

(1) Lab9101, ex Roquette-Freres, France
(2) Ascend ™, Cargill, USA
(3) ex Firmenich, Ref. 050001 OB21000
(4) Yelkin SS, ex ADM

A syrup was prepared using the trehalose, hydrogenated starch hydrolysate, ascorbic acid and water. The mixture was heated to 125° to reduce the moisture content of the syrup. The Tg was 38° C. The orange oil and lecithin were mixed with the concentrated syrup with high shear to form a uniform melt which was then extruded under pressure through a die plate with 0 8 mm diameter holes into a cold solvent for chilling and breaking of the strands. After drying, 0 5% silicon dioxide was added as free flow agent. The resulting granular product had a glass transition temperature of 53° C. and a clear, white appearance indicating an absence of browning reactions (e.g. caramelization).

### Example 2

### Preparation of a Comparative Granular Delivery System

A granular delivery system was prepared using the following ingredients in the amounts shown:

TABLE 2

| Ingredient | Grams |
|---|---|
| Hydrogenated Starch Hydrolysate (1) | 1300 |
| Sucrose (2) | 1300 |
| Ascorbic Acid | 30 |
| Cold Pressed Orange Oil (3) | 300 |
| Soya lecithin (4) | 30 |
| Water | 700 |

(1) Lab9101, ex Roquette-Freres, France
(2) Pure Cane Extra Fine Granulated Sugar, ex Domino Foods
(3) ex Firmenich, Ref. 050001 OB21000
(4) Yelkin SS, ex ADM

A syrup was prepared using the sugar, hydrogenated starch hydrolysate, ascorbic acid and water. The mixture was heated to 126° to reduce the moisture content of the syrup. The Tg was −3° C. The orange oil and lecithin were mixed with the concentrated syrup with high shear to form a uniform melt which was then extruded under pressure through a die plate with 0.8 mm diameter holes into a cold solvent for chilling and breaking of the strands. The resulting product could not be dried further as a result of its extremely low Tg and the caking of the viscous mass. The material had a dark brown indicating browning reactions (e.g. caramelization) occurring during the process.

### Example 3

### Preparation of a Comparative Granular Delivery System

A granular delivery system was prepared using the following ingredients in the amounts shown:

TABLE 3

| Ingredient | Grams |
|---|---|
| Maltodextrin 18 DE (1) | 1300 |
| Trehalose dihydrate (2) | 1422 (1300 on an anhydrous basis) |
| Ascorbic Acid | 30 |
| Cold Pressed Orange Oil (3) | 300 |
| Soya lecithin (4) | 30 |
| Water | 700 |

(1) StarDri 18, ex Tate and Lyle
(2) Ascend ™, ex Cargill, USA
(3) ex Firmenich, Switzerland (ref. 050001 OB21000)
(4) Yelkin SS, ex ADM

A syrup was prepared using the trehalose, maltodextrin and water. The mixture was heated to 127° to reduce the moisture content of the syrup. The Tg was 30° C., and moisture content was 8.1%. The orange oil and lecithin were mixed with the concentrated syrup with high shear to form a uniform melt which was then extruded under pressure through a die plate with 0.8 mm diameter holes into a cold solvent for chilling and breaking of the strands. After drying, 0.5% silicon dioxide was added as free flow agent. The resulting product contained 5.7% moisture and had a glass transition temperature of 54° C. The strands were had a light brown appearance due to browning reactions (e.g. caramelization).
Examples 1 and 3 demonstrate that, the hydrogenated starch hydrolysate-trehalose matrix is less susceptible to reducing reactions such as browning, e.g. through caramelization.

1.-8. (canceled)

9. A granular, extruded delivery system comprising a glassy carbohydrate matrix and an active ingredient encapsulated therein, the matrix comprising trehalose and a hydrogenated starch hydrolysate having number average degree of polymerization, DPn, of between 5 and 100, or a number average molecular weight, Mn, of between 800 and 16000 Da.

10. The delivery system according to claim 9, wherein the hydrogenated starch hydrolysate is present in an amount of from 90 to 35% by weight, based on the total weight of the matrix.

11. The delivery system according to claim 10, wherein the trehalose is present in an amount of from 10 to 65% by weight, based on the total weight of the matrix.

12. The delivery system according to claim 9, wherein the trehalose is present in an amount of from 10 to 65% by weight, based on the total weight of the matrix.

13. The delivery system according to claim 9, wherein the trehalose and hydrogenated starch hydrolysate are present essentially in a 1:1 ratio calculated in an anhydrous state.

14. The delivery system according to claim 9, wherein the matrix further comprises an antioxidant.

15. The delivery system according to claim 14, wherein the antioxidant is ascorbic acid.

16. A method of preparing a granular delivery system which comprises:
creating a melt emulsion comprising a continuous phase and a dispersed phase, the continuous phase comprising trehalose and a hydrogenated starch hydrolysate having a number average degree of polymerization, DPn, of between 5 and 100, or a number average molecular weight, Mn, of between 800 and 16000 Da, and the dispersed phase comprising an active ingredient,
forcing the melt emulsion through a die or orifice to form an extrudate,
cooling and granulating the extrudate, and
optionally drying the granules.

17. The method according to claim 16, wherein the hydrogenated starch hydrolysate is present in an amount of from 90 to 35% by weight, based on the total weight of the matrix.

18. The method according to claim 16, wherein the trehalose is present in an amount of from 10 to 65% by weight, based on the total weight of the matrix.

19. The method according to claim 16, wherein the trehalose and hydrogenated starch hydrolysate are present essentially in a 1:1 ratio calculated in an anhydrous state.

20. A granular delivery system formed by the method of claim 9.

21. A foodstuff, a beverage, an edible composition, a pharmaceutical composition, a nutraceutical composition, a chewing-gum or a toothpaste, comprising the granular delivery system of claim 20.

22. The foodstuff according to claim 21 having a pH of 6 or less.

23. A method of encapsulating an active ingredient which comprises extruding a melt emulsion a continuous phase and a dispersed phase, the continuous phase comprising trehalose and a hydrogenated starch hydrolysate having a number average degree of polymerization, DPn, of between 5 and 100, or a number average molecular weight, Mn, of between 800 and 16000 Da, and the dispersed phase comprising an active ingredient, wherein the trehalose and hydrogenated starch hydrolysate are present in relative amounts such that a matrix is extruded having a full glassy state for optimal encapsulation of the active ingredient.

24. The method of claim 23 wherein the hydrogenated starch hydrolysate is present in an amount of from 90 to 35% by weight and the trehalose present in an amount of from 10 to 65% by weight.

25. The method of claim 23 which further comprises providing the delivery system in a foodstuff, a beverage, an edible composition, a pharmaceutical composition, a nutraceutical composition, a chewing-gum or a toothpaste.
